(19) United States
(12) Patent Application Publication
(10) Pub. No.: US 2020/0268374 A1
(43) Pub. Date: Aug. 27, 2020

(54) DEVICE FOR SUTURING A HEART VALVE PROSTHESIS

(71) Applicants: Marco Vola, Bron (FR); CMI'NOV, Monistrol-Sur-Loire (FR)

(72) Inventors: Marco Vola, Bron (FR); Bernard Pain, Monistrol-Sur-Loire (FR)

(21) Appl. No.: 16/648,790

(22) PCT Filed: Aug. 10, 2018
(86) PCT No.: PCT/FR2018/052054

§ 371 Date: Mar. 19, 2020

(30) Foreign Application Priority Data
Oct. 9, 2017 (FR) 1759440

(51) Int. Cl. A61B 17/04; A61B 17/062

(52) U.S. Cl. CPC A61B 17/0483; A61B 2017/00243; A61B 17/062

(57) ABSTRACT

A device for suturing a heart valve prosthesis includes an assembly cooperating with an actuation handle. The assembly is arranged at the free end of a manipulation rod and includes a set of needles arranged in parallel and pointing in a direction opposite the free end of the rod and connected in pairs by suture threads running along and inside the rod. The set of needles is maintained in a cylindrical configuration between a plurality of guides through which the pairs of needles slide freely, and a plurality of pusher elements supporting the pairs of needles from the direction of the free end of the rod. The guides and the pusher elements are movable along the rod and are connected pivotably to the rod so as to change, under the action of control means, from a position retracted along the rod to a deployed position spaced apart from the rod.

### TECHNICAL FIELD

The present disclosure relates to the technical field of cardiac surgery and relates more specifically to a heart valve suturing device, e.g. for repairing or replacing an aortic valve or a mitral valve in a heart.

### BACKGROUND

In the cardiac surgery field, it is known to carry out open-heart aortic or mitral valve replacement interventions, i.e. in extracorporeal circulation. The valve implant is attached via a plurality of sutures put in place after the calcified valve is ablated. To do so, needles and suture thread must be used to create a thread bundle of up to 40 threads, enabling the valve prosthesis to be positioned and attached to the valve's native annulus. In practice, these threads are placed one by one and are spaced out evenly around the circumference of the native annulus.

The disadvantage of this technique results from the fact that the surgeon must handle a large quantity of threads which leave the patient's open heart, without mixing or tangling them up. In practice, the threads are spread all around the opening made in the thorax, in order to tie them together two by two later to attach the valve prosthesis. This operation is a long and fastidious one and features risks, notably during the delicate manipulation of the suture threads.

### SUMMARY OF THE DISCLOSURE

The purpose of the disclosed embodiments is to resolve the disadvantages involved in the state of the art, by proposing a cardiac valve prosthesis suturing device, which enables a surgeon to handle the suture threads with the risk of mixing, losing or tangling them.

One other objective is therefore to provide this device which enables the time needed for the surgical intervention to be considerably shortened.

Therefore, a cardiac valve prosthesis suturing device has been developed, which is remarkable in that it comprises an assembly which is able to cooperate with a handle subjected to control means for actuating the assembly to suture the prosthesis to a cardiac valve. The assembly is arranged at the end of a manipulation rod and comprises:
a set of needles arranged parallel to one another and pointing in a direction opposite the free end of the rod and being connected in pairs by suture threads running along and inside the rod, the set of needles is maintained in a cylindrical configuration between, on the one hand, a plurality of guides through which the pairs of needles slide freely, and, on the other hand, a plurality of pusher elements supporting the pairs of needles from the direction of the free end of the rod, the guides and the pusher elements are movable along the rod and are connected pivotably to the rod so as to change, under the action of the control means, from a position retracted along the rod to a deployed position spaced apart from the rod in order to increase the diameter of the cylindrical configuration of the set of needles;
two retention rings intended to cross the needles in order to maintain the latter in the cylindrical configuration, the rings comprising blocking means for blocking said rings in position around the needles.

In this way, the device enables a surgeon to place the free end of the manipulation rod through the cardiac valve to be repaired and to position all the needles in the cylindrical configuration supported under the native annulus.

Whereupon the needles are supported under the native annulus, one of the retention rings is positioned in counter support on the other side of the native annulus to enable the pusher elements to push the needles until they cross the native annulus and are inserted into the retention ring. The ring blocking means enable all the needles to be maintained in cylindrical configuration to enable the pusher elements, the guides and more generally the manipulation rod to be removed from the heart.

In this configuration, the surgeon can remove all the needles held by the retention ring from the heart simultaneously to form a bundle of suture threads attached on one side to the valve's native annulus and on the other connected to the different needles.

This thread bundle is controlled, and so the risks of losing, mixing or tangling the suture threads are eliminated.

The surgeon may then position the valve prosthesis, e.g. a textile ring, directly on the retention ring, which is pierced by all the needles, and blocked in counter support by the second retention ring which is inserted around the needles in counter support.

In this configuration, the surgeon may remove the lower retention ring, i.e. the one positioned beside the cardiac valve, enabling them to slide the valve prosthesis to the cardiac valve's native annulus and in counter support against it. At this stage, the surgeon only needs to remove the needles two by two and tie off the suture threads to attach the valve prosthesis definitively. The operation is quick and easy.

According to a specific production form, each guide is presented as a sleeve comprising an eight-shaped cross section, and enabling a pair of needles to be guided in the optimum manner, while enabling the suture threads to pass through whereupon the guide is removed.

In the same way, each pusher element is presented, e.g., as a sleeve comprising an eight-shaped cross section, with in particular a bottom wall to be able to push the pair of needles in the direction of the valve, with this bottom wall also presenting an opening for the suture thread to pass through whereupon the pusher element is removed.

According to a specific production form, each retention ring comprises:
an external ring pierced with a plurality of openings evenly distributed in pairs around the circumference of the external ring, each opening being open and opening onto the edge of the external ring, in particular to enable the suture threads to pass through whereupon the ring is removed, or whereupon the needles are removed;
an internal ring mounted coaxially in the external ring and with the capacity to rotate in relation to the external ring, the internal ring enables portion walls of this internal ring to block at least part of the openings, and to house in complementary notches created in the needles to block the ring and maintain all the needles in cylindrical configuration.

Preferably, the internal ring comprises a serrated internal periphery to cooperate with the means that enable it to rotate.

According to a specific production form, the pusher elements are each connected to the manipulation rod by at least one arm articulated in relation to the rod.

Advantageously, the arms of the guides and the pusher elements are connected pivotably to the rod in such a way as to change, under the action of the control means, simultaneously or selectively and independently from each other, from the folded position to the deployed position.

Moreover, the device comprises advantageously a protective cover mounted sliding around the manipulation rod to cover the pointed end of the set of needles.

### BRIEF DESCRIPTION OF THE DRAWINGS

Further characteristics and advantages of the disclosed embodiments will become apparent from the description provided below, which is for reference only and is in no way restrictive, with reference to the accompanying figures, wherein:

FIG. 1 depicts a perspective illustration of the device, with the protective cover removed and a retention ring;

FIG. 2 depicts a perspective illustration of the retention ring, with the internal ring not in blocking position;

FIG. 3 is similar to FIG. 2, with the internal ring in blocking position;

FIG. 4 illustrates the device in a longitudinal section, after insertion through the cardiac valve;

FIG. 5 is similar to FIG. 4, with the protective cover removed and the guides and pusher elements moved in the direction of the cardiac valve;

FIG. 6 is similar to FIG. 5, with the guides and the pusher elements pivoted to move away from the rod in order to position the needles in support under the cardiac valve's native annulus;

FIG. 7 is similar to FIG. 6, with one of the retention rings having been placed in counter support on the other side of the cardiac valve's native annulus;

FIG. 8 is similar to FIG. 7, with the pusher elements having been moved in the direction of the guides for the needles to cross the mitral valve's native annulus and the retention ring;

FIG. 9 is similar to FIG. 8, with the retention ring having been pivoted to block all the needles in their cylindrical configuration;

FIG. 10 is similar to FIG. 9, with the pusher elements moved to separate from the guides for their withdrawal;

FIG. 11 is similar to FIG. 10, with the guides moved in the direction of the pusher elements for their withdrawal;

FIG. 12 is similar to FIG. 11, with the manipulation rod removed to leave only the set of needles maintained in a cylindrical configuration by the retention ring;

FIG. 13 is similar to FIG. 12, with all the needles removed to form a bundle of suture threads attached to the cardiac valve's native annulus;

FIG. 14 is similar to FIG. 13, before a cardiac valve prosthesis is anchored;

FIG. 15 is similar to FIG. 14, the valve prosthesis is anchored on the set of needles until it is in counter support against the retention ring;

FIG. 16 is similar to FIG. 15, with a second retention ring in counter support on the first to surround the cardiac valve prosthesis and this second retention ring in blocking position to maintain all the needles in a cylindrical configuration;

FIG. 17 is similar to FIG. 16; with the first retention ring having been removed;

FIG. 18 is similar to FIG. 17, with the cardiac valve prosthesis slid in the direction of the cardiac valve's native annulus;

FIG. 19 is similar to FIG. 18, with the cardiac valve prosthesis positioned in counter support against the cardiac valve's native annulus, pending attachment by knotting the plurality of suture threads.

### DETAILED DESCRIPTION

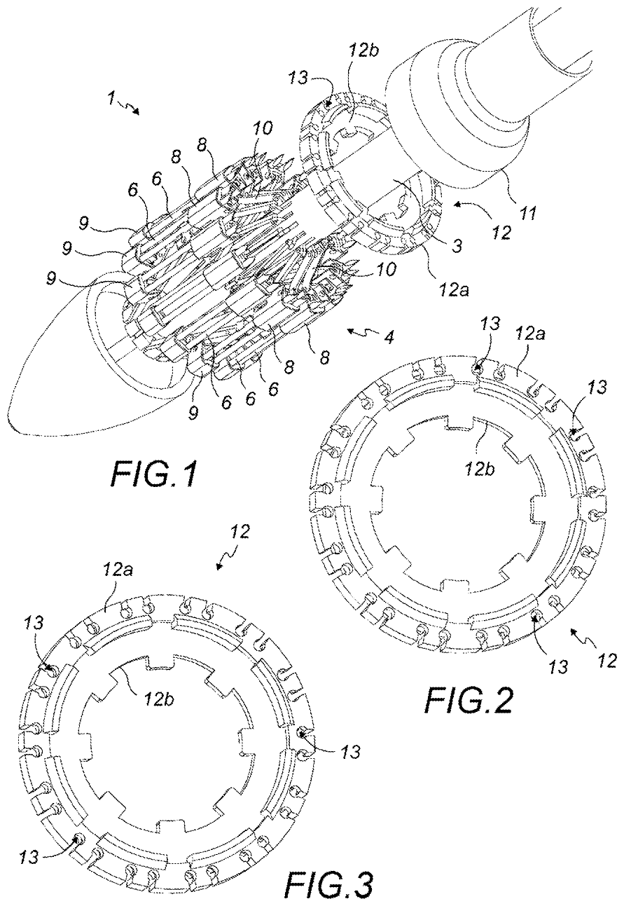

Referring to FIG. 1, the present disclosure relates to a device (1) used to attach a cardiac valve prosthesis (2) with sutures, e.g. an aortic valve or a mitral valve. The device (1) is intended to penetrate the cardiac valve (5) to be repaired, through the chest cavity and in open heart surgery.

Therefore, the device (1) comprises a manipulation rod (3) at the end of which if an assembly (4) to suture the prosthesis (2) on the valve's native annulus (5). The valve prosthesis (2) may be of any type, such as reinforcement ring, e.g. made of textile. The other end of the rod (3) is intended to cooperate with a handle, not shown, under the control of control means, not shown, e.g. in the form of a trigger, to operate the assembly (4).

The device (1) enables a plurality of simultaneous sutures to be carried out, around the circumference of the valve's native annulus (5) in order to enable the prosthesis (2) to be attached. The device (1) comprises a set of needles (6) maintained in a cylindrical configuration and linked two by two by suture threads (7). More precisely, the needles (6) are arranged parallel to each other, at the end of the manipulation rod (3), pointing towards a direction opposite the free end of the rod (3). The suture threads (7) which connect the needles (6) two by two, run along and inside the rod (3). The set of needles (6) is maintained in the cylindrical configuration, on the one hand, by a plurality of guides (8) and, on the other hand, by a plurality of pusher elements (9).

The guides (8) are arranged to surround and hold the pointed end of each pair of needles (6). In other words, two needles (6) are placed inside the same guide (8). The guides (8) and needles (6) slide freely in relation to each other. Each guide (8) is presented e.g. as a sleeve comprising an eight-shaped cross section to enable the needles (6) to be maintained in the optimum manner, while enable it to slide along them. Each guide (8) is open at both ends to let the suture thread (7) escape whereupon the guide (8) is removed.

On the other side, the needles (6) are maintained in pairs by a plurality of pusher elements (9), e.g. in the form of sleeves, comprising, in the same way, an eight-shaped cross section, in particular with a bottom wall enabling each pair of needles (6) to be pushed. The bottom wall presents an opening for the suture thread (7) to pass through whereupon the pusher element (9) is removed. Each pusher element (9) acts as an adapted base to maintain the non-pointed end of the needles (6) and to provide axial thrust force on these needles (6).

The guides (8) and the pusher elements (9) are subjected to the manipulation rod (3) via articulated arms (10) mounted pivoting in relation to the rod (3), under the action of the control means. Via these arms (10), the guides (8) and the pusher elements (9) can be moved along the rod (3), still under the action of the control means, and may adopt a folded position along the rod (3) or a deployed position away from the rod (3), in particular to increase the diameter of the cylindrical configuration of the set of needles (6). Preferably, the guides (8) and the pusher elements (9) are connected pivotably to the rod (3) in such a way as to change, under the action of the control means, simultaneously or selectively and independently from each other, from the folded position to the deployed position.

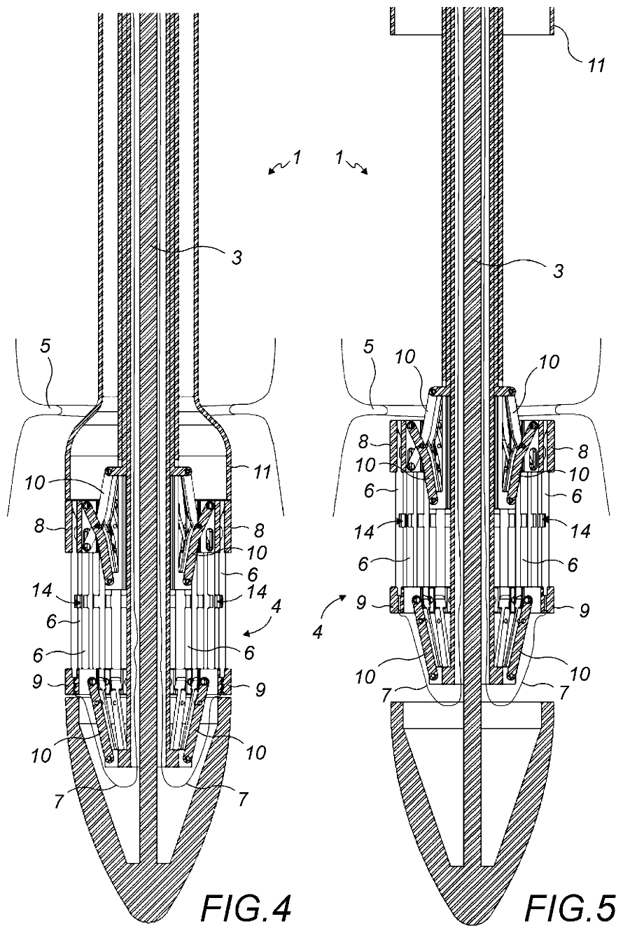

Referring to FIGS. 1 and 4, the device (1) also comprises a protective cover (11) mounted sliding around the rod (3) to cover the pointed end of the set of needles (6) and the plurality of guides (8).

Therefore, during the surgical operation, the free end of the manipulation rod (3) is pushed through the cardiac valve (5), then the protective cover (11) is slid to be removed.

Referring to FIG. 5, the plurality of guides (8) and pusher elements (9) are moved along the rod (3), under the action of the control means, and in particular in a direction opposite to the free end of the rod (3) so that the pointed ends of the needles (6) are close to and under the native annulus of the valve (5) to be repaired.

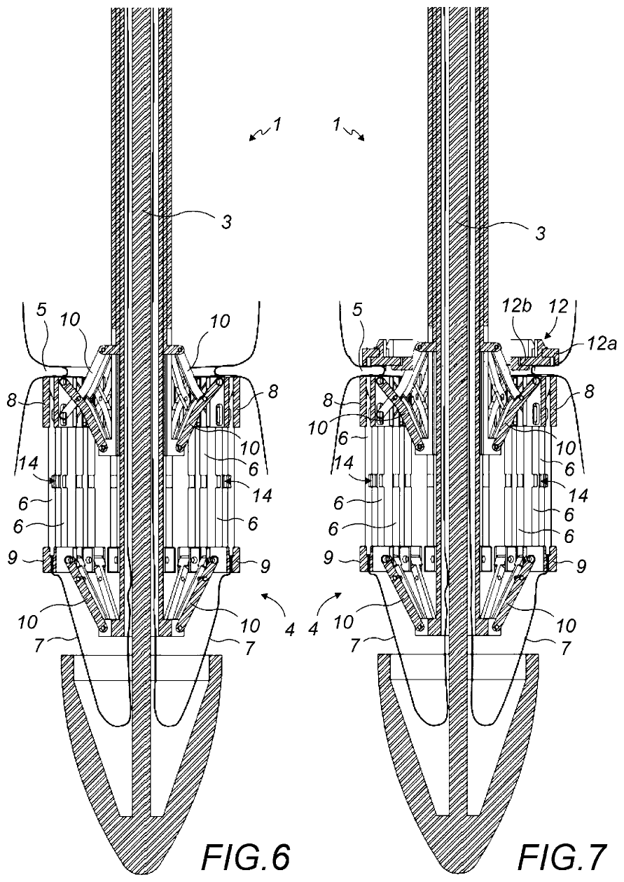

Referring to FIG. 6, the control means are actuated to separate the guides (8) and the pusher elements (9) in deployed position via the pivoting arms (10). This enables the diameter of the cylindrical configuration of the set of needles (6) to be adjusted to the diameter of the native annulus of the valve (5) to be repaired.

Referring to FIG. 7, whereupon the device (1), and in particular all the guides (8) are in support under the valve (5), a first retention ring (12) is slid along the rod (3) to be in counter support on the plurality of guides (8), on the other side of the valve (5).

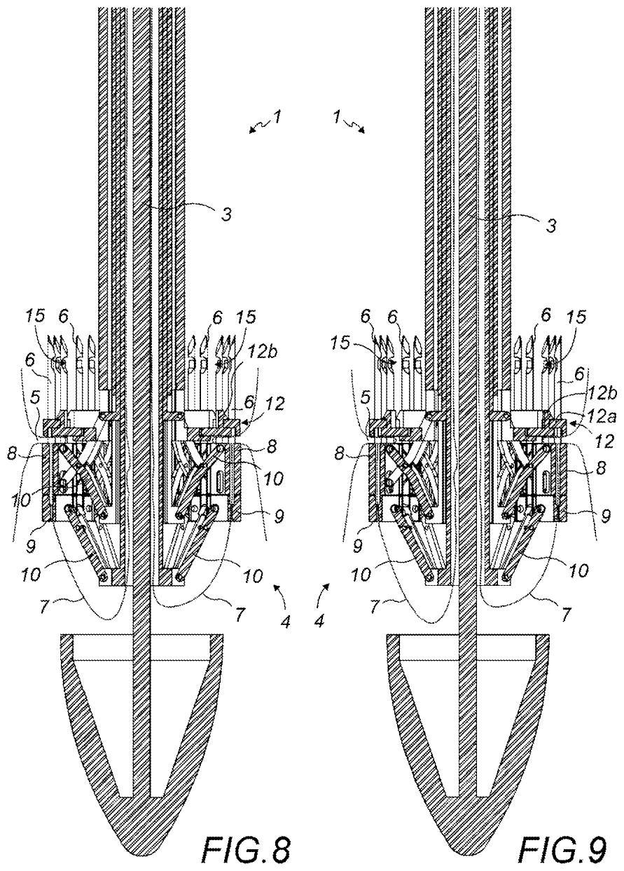

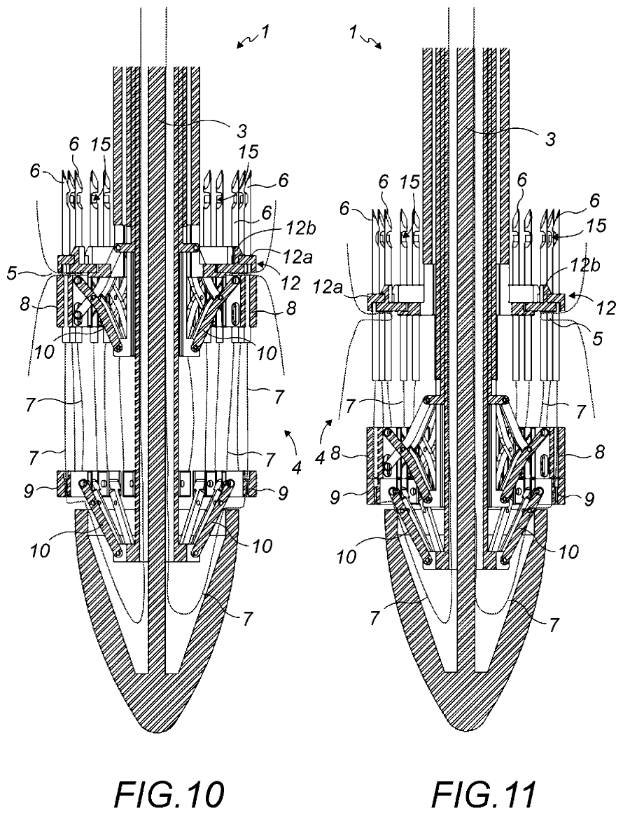

In this configuration, and referring to FIG. 8, the surgeon is able to actuate the control means to move the pusher elements (9) in the direction of the guides (8). In this way, the set of needles (6) is pushed in the direction of the native ring until the valve (5) is pierced and is inserted through the first retention ring (12).

To this end, and referring to FIGS. 2 and 3, the retention ring (12) comprises an external ring (12a) pierced with a plurality of openings (13) distributed evenly in pairs around its circumference. In particular, these openings (13) enable each pair of needles (6) to be inserted. Each opening (13) is open and opens onto the edge of the external ring (12a), to enable the retention ring (12) and the suture threads (7) to be removed later. The first ring (12) also comprises blocking means as an internal ring (12b), mounted coaxially in the external ring (12a), with the capacity to rotate in relation to the external ring (12a). In particular, the rotation of the internal ring (12b) enables wall portions of this internal ring (12b) to block at least part of the openings (13) in the external ring (12a).

In this way, and referring to FIG. 11, whereupon the internal ring (12b) is pivoted, the wall portions block the openings (13) and house in the first complementary notches (14) that each needle (6) comprises. This enables the ring (12) to be clocked and the set of needles (6) to be maintained in cylindrical configuration.

At this stage, the surgeon may actuate the control means to move the guides (8) and the pusher elements (9) along the rod (3) and in the direction of its free end. In this way, the arms (10) which link the guides (8) and the pusher elements (9) to the rod (3) may be pivoted in a folded position along the rod (3), for the manipulation rod (3) to be removed.

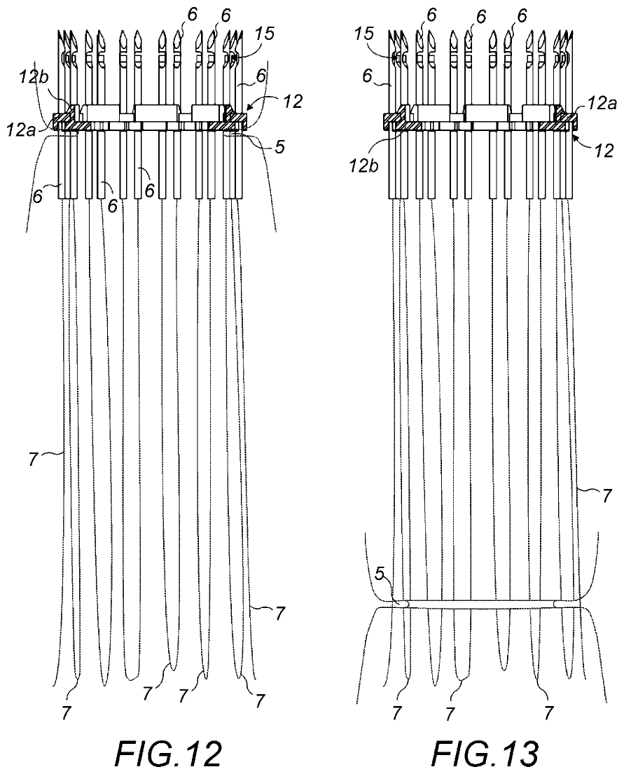

Referring to FIG. 12, whereupon the manipulation rod (3) is removed it releases the suture threads (7) which run up along and inside this rod (3) to leave the bundle of threads (7) attached in pairs to the set of needles (6) maintained in a cylindrical configuration through the valve (5) by the retention ring (12).

Referring to FIG. 13, the surgeon can now remove the set of needles (6) which forms a rigid assembly extended by a bundle of suture threads (7) attached to the cardiac valve (5). In this way, the suture threads (7) in the form of a bundle, cannot be lost, mixed or tangled. The suture is quick and is performed simultaneously by all the needles (6) and not one by one as in the state of the art.

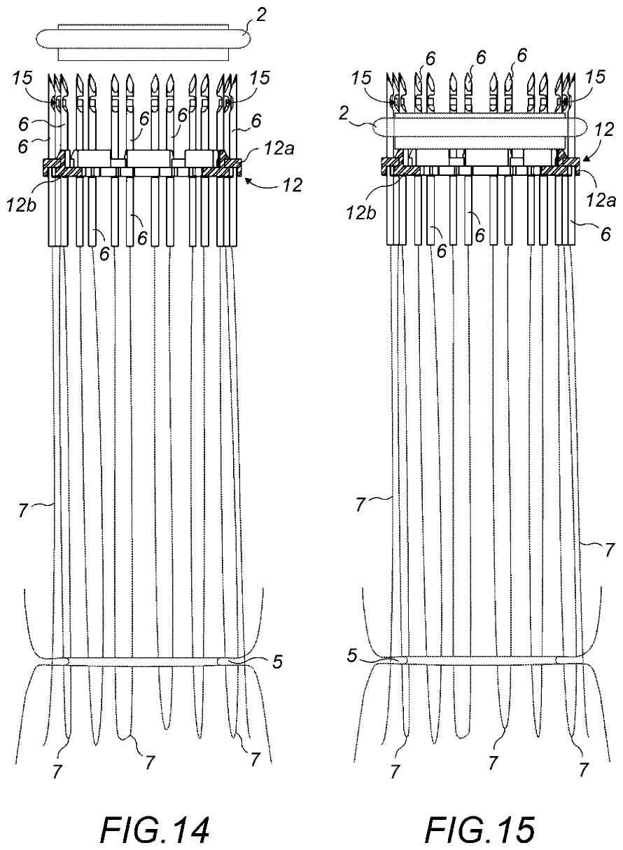

Referring to FIG. 14, the surgeon can then position the valve prosthesis (2) against the retention ring (12) pierced by the set of needles (6) with a view to later attachment.

Referring to FIG. 15, a second retention ring (12) is positioned in support against the valve implant, and in particular in counter support on the first retention ring (12). This second retention ring (12) is identical to the first ring and also comprises blocking means in the form of an internal ring (12b) which is adapted to pivot and block in the second complementary notches (15) that the needles (6) have.

Referring to FIGS. 2 and 3, the internal periphery of the internal ring (12b) of the retention rings is serrated in order to enable a specific complementary tool to be inserted to rotate this internal ring (12b). The specific tool is of the appropriate type.

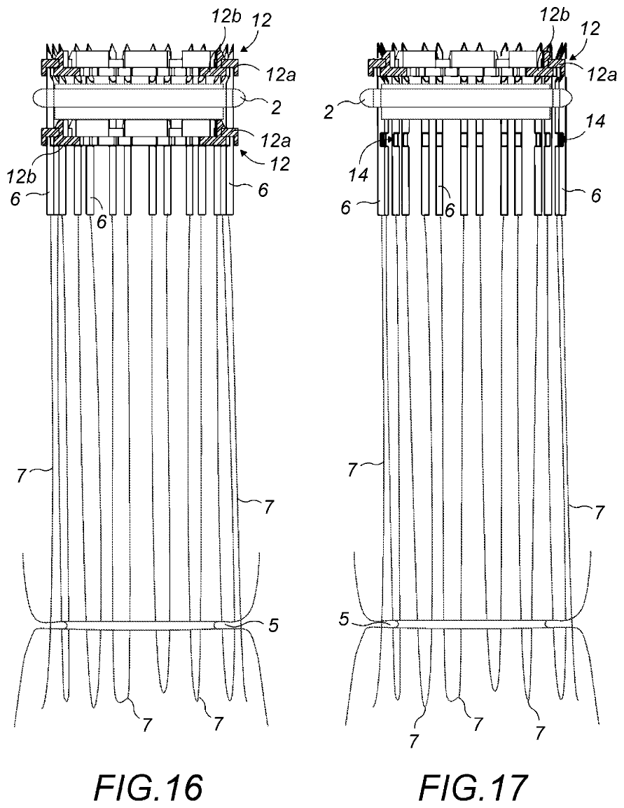

Referring to FIG. 16, whereupon the second retention ring (12) is locked, it enables the set of needles (6) to be maintained in the cylindrical configuration. In this way, the first retention ring (12) placed on the side of the cardiac valve (5) may be released by rotating its internal ring (12b) and removed, allowing the suture threads (7) to escape through the openings (13) through to the external ring section (12a).

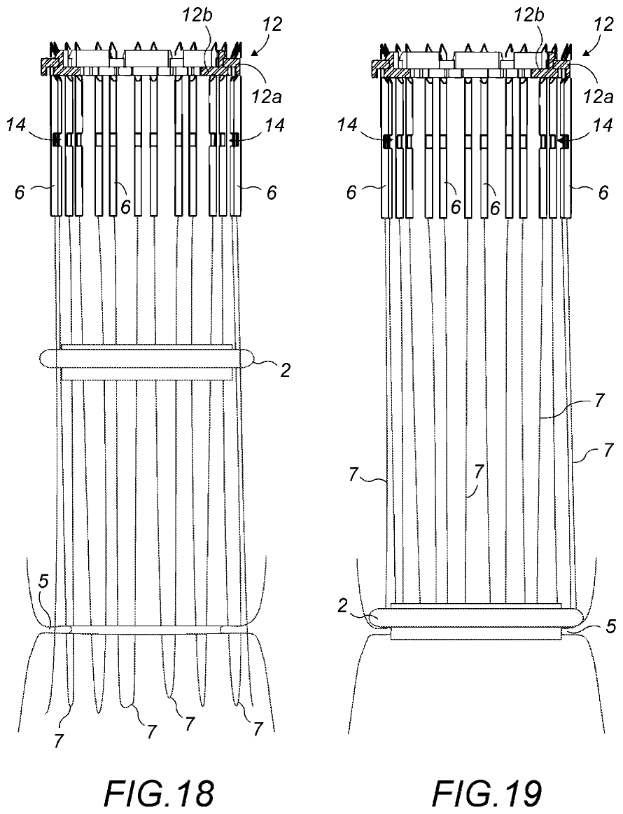

Referring to FIGS. 17, 18 and 19, the valve prosthesis (2) is then slid along the needles (6) and the suture threads (7) until it is in support against the native annulus of the cardiac valve (5) to be repaired. In this configuration, the suture threads (7) may be retrieved and tied off two by two to attach the prosthesis (2) definitively to the cardiac valve (5).

If follows from the above that the disclosed embodiments provide an advantageous device (1) in that it enables simultaneous sutures to be performed through the cardiac valve (5), then all the suture threads (7) to be removed simultaneously as a bundle, which prevents these threads (7) from being mixed, lost or tangled, with a view to them being tied off two by two. The resulting surgical operation is significantly improved in terms of efficacy and speed.

1. A device for suturing a heart valve prosthesis, the device comprising: an assembly for cooperating with a handle subjected to control means for actuating the assembly in order to suture the prosthesis to a heart valve, said assembly is arranged at the free end of a manipulation rod and comprises:
a set of needles arranged parallel to one another and pointing in a direction opposite the free end of the rod, and being connected in pairs by suture threads running along and inside the rod; the set of needles is maintained in a cylindrical configuration between, on the one hand, a plurality of guides through which the pairs of needles slide freely, and, on the other hand, a plurality of pusher elements supporting the pairs of needles from the direction of the free end of the rod, the guides and the pusher elements are movable along the rod and are connected pivotably to the rod so as to change, under the action of the control means, from a position retracted along the rod to a deployed position spaced apart from the rod in order to increase the diameter of the cylindrical configuration of the set of needles;
two retention rings intended to be crossed by the set of needles in order to maintain the latter in the cylindrical configuration, the rings comprising blocking means positioned around the needles.

2. A device according to claim 1, wherein each guide is presented as a sleeve comprising an eight-shaped cross section.

3. A device according to claim 1, wherein each pusher element is presented as a sleeve comprising an eight-shaped cross section, with a bottom wall to push the pair of needles, and with said bottom wall presenting an opening for the suture thread to pass through.

4. A device according to claim 1, wherein each retention ring comprises:
an external ring pierced with a plurality of openings evenly distributed in pairs around the circumference of the external ring, with each opening open and through to the section of the external ring;
an internal ring mounted coaxially in the external ring and with the capacity to rotate in relation to the external ring, the rotation of the internal ring enables wall portions to block at least part of the openings, and to house in complementary notches created in the needles to block the ring and maintain all the needles in cylindrical configuration.

5. A device according to claim 4, wherein the internal ring comprises a serrated internal periphery to cooperate with the means that enable it to rotate.

6. A device according to claim 1, wherein the guides and the pusher elements are each connected to the rod by at least one arm articulated in relation to the rod.

7. A device according to claim 6, wherein the arms of the guides and the pusher elements are connected pivotably to the rod in such a way as to change, under the action of the control means, simultaneously or selectively and independently from each other, from the folded position to the deployed position.

8. A device according to claim 1, further comprising a protective cover mounted sliding around the rod to cover the pointed end of the set of needles.
